(19) United States
(12) Patent Application Publication
(10) Pub. No.: US 2003/0124178 A1
(43) Pub. Date: Jul. 3, 2003

(54) Soft, adherent, soluble oral patch

(76) Inventor: Jeffrey T. Haley, Mercer Island, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 10/287,843

(22) Filed: Nov. 5, 2002

Related U.S. Application Data
(63) Continuation-in-part of application No. 10/236,289, filed on Sep. 4, 2002, now abandoned.
(60) Provisional application No. 60/344,577, filed on Dec. 28, 2001.

(51) Int. Cl. A61K 9/70; A61K 9/20

(52) U.S. Cl. 424/449

(57) ABSTRACT

A soft, adherent, soluble oral patch for delivering topical medication in the mouth including a hydrophilic polymer that is liquid at human mouth temperatures. Preferably, the hydrophilic polymer gels to a solid at temperatures just below human mouth temperatures. The structure of the oral patch is formed with a porous network that remains solid at human mouth temperatures and slowly dissolves in saliva. In some embodiments, the network is elasto-plastic and in some cases it is hydrophilic. The hydrophilic polymer is located within pores of the network, along with a desired medication. The oral patch is formed by mixing and hydrating the ingredients, bringing them to a hot temperature just below boiling, and cooling them to form a gel. The hot mixture may be poured into molds and the mold may also serve as packaging for delivery. The packaging may comprise a germ barrier moisture passing film which allows the oral patch to dry out or become re-hydrated without growing mold.

### BACKGROUND

For treatment of health problems in the mouth or throat, people have for centuries held in their mouths a composition containing herbal or other medication for topical application. The oldest name for such a composition, derived from Latin and previously from Greek, is “troche”. A modem form of troche is the cough drop, so named because it was formed by “dropping” hot, viscous, sugar-based candy onto a sheet or into a mold where it cools to form the troche. Another modem form of troche is the throat “lozenge”, so named because it was in the shape of a diamond (like on playing cards), which is the meaning of the word “lozenge”. The structural characteristics of these types of troches are determined by their primary structural ingredients which are typically corn syrup or sugars, including sugar alcohols. The troches are relatively hard and are often irritating to tender surfaces such as canker sores. These troches are only mildly adherent to teeth and not significantly adherent to gums, cheeks, or lips.

To achieve higher concentrations of medication at a particular spot in the mouth than troches can deliver, adherent oral patches have been developed. An oral patch typically includes one or more flexible saliva flow impeding layers that do not dissolve entirely such as invented by Anthony et al. and disclosed in U.S. Pat. No. 5,713,852. Another example of an oral patch is the DentiPatch which has one or more non-soluble thermo-plastic layers and lidocaine, offered for sale by Noven Pharmaceuticals, Inc. As used herein, the word “patch” does not include preparations that move about the mouth rather than resting in one place, such as cough drops, throat lozenges, or other troches, and therefore do not impede local flow of saliva. Nor does it include preparations that do not hold together as a single item when held in the mouth such as preparations of powder, liquid, paste, viscous liquid gel, or a tablet or troche that crumbles into a powder or paste when chewed or placed in saliva.

The most significant differences between an oral patch as used herein and other forms of medicinal preparations such as troches are that an oral patch is designed to (1) release medication into the mouth over a relatively long period of time, such as 30 minutes or more, (2) be at least mildly adherent and adequately conforming to adhere so that it can be placed in a preferred location and not be dislodged by gravity or gentle movement, (3) restrict local flow of saliva so that the medication can reach high concentrations along side the patch, and (4) remain in the mouth as a single item that will not spread to be in a plurality of locations in the mouth at one time.

There are many uses for preparations containing a medication to be delivered topically in the mouth. In many treatment situations, it is advantageous to retain the preparation at one location in the mouth rather than allowing it to move in the mouth such as when talking. U.S. Pat. No. 6,139,861 issued to Mark Friedman surveys the known methods for adhering a slowly dissolving medication to a location within the mouth. These methods include two forms of adherent soluble patches, referred to by Friedman as “mucoadhesive erodible tablets”. These tablets are formed using polymers carboxymethylcellulose, hydroxymethylcellulose, polyacrylic acid, and carbopol-934. None of these polymers melts to a liquid at human mouth temperatures.

### SUMMARY OR THE INVENTION

The invention is a soft, adherent oral patch including a hydrophilic polymer that is liquid at human mouth temperatures. The oral patch is made of two primary components.

The first component is a porous molecular network formed as a unitary solid structure that remains a solid at human mouth temperatures. In preferred embodiments, this network is elasto-plastic (elastic or plastic or a combination thereof), giving it a soft feel in the mouth, in contrast to being crumbly or a paste. The network is preferably hydrophilic so that, even when applied to a wet mucosal surface in the mouth, it will tend to adhere by absorbing moisture from the mucosal surface. Preferably, the network slowly dissolves in saliva so that the patch merely dissipates over time and the patch never has to be removed form the mouth.

The second component is a hydrophilic polymer that is liquid at human mouth temperatures distributed throughout the pores of the network. Because the polymer is hydrophilic and liquid at human mouth temperatures, it will adhere very well to wet surfaces inside the mouth and is quite soft which provides a soothing feeling to any sensitive tissue such as canker sores. The adherent oral patch will adhere to teeth, gums, cheek, lips, or tongue without the user first drying saliva from the tissue. If the patient merely places the oral patch in his or her mouth and holds it in the desired location for 10 to 40 seconds, it will then adhere to the tissues that it has been touching without movement, even though those tissues are wet. This is far easier for patients to use than requiring that the tissue first be dried with a towel before the adherent oral patch is placed. If the patient wants to use an oral patch in the lip or under the tongue, the oral patch can easily be removed for talking and then easily be replaced without using a towel or a mirror.

A desired medication is also located within the pores of the network along with the hydrophilic polymer.

The network component may be comprised of a thermo gel having a melting temperature higher than human mouth temperatures. Preferably, the thermo gel is elasto-plastic, such as formed by a mixture of the hydrogels konjac gum and xanthan gum dissolved in hot water and then cooled to form an elasto-plastic gel. Alternatively, the network may be comprised of a complex carbohydrate, such as cellulose, pectin, maltodextrin, or starch from potato, rice, corn, or wheat. Also, the network may be comprised of a hydrogel with a melting temperature higher than temperatures in the human mouth formed of amino acids, such as peptides.

In preferred embodiments, the hydrophilic polymer gels to a solid at room temperatures. This allows the oral patch to be removed from the mouth and placed on a smooth surface, such as a plastic bag. Because the hydrophilic polymer then gels, the oral patch again becomes handleable with the fingers to return it to the mouth without being too sticky to handle or leaving a residue on the fingers or on the plastic bag. In one such embodiment, the hydrophilic polymer is protein gelatin rendered from animal tissue, which solidifies at just below mouth temperatures and remains a solid even at clothes pocket temperatures so it will not melt in a pocket.

In another aspect, the invention is a method for manufacturing an adherent oral patch. In this method, ingredients for forming the porous network, molecules of the hydrophilic polymer, molecules of the medication, and water are mixed together. The mixture is heated to dissolve all ingredients, either before the ingredients are added together or after they are added together, and the mixture is then cooled, thereby causing the ingredients for forming a network to form the porous network as a unitary solid structure having the medication and the hydrophilic polymer within its pores. Before it is cooled, the hot mixture may be deposited into a mold of a suitable shape to form the preferred unitary solid structure. The mold may be formed in powdered starch, as is well known in the candy making industry for forming gumdrops. Alternatively, the mold may be formed of a rigid material such as metal or plastic. If the mold is thin plastic or aluminum, it may also serve as packaging for delivery of the oral patch to the consumer.

### BRIEF DESCRIPTION OF THE DRAWING

FIG. 1a shows a side view of a soft, adherent, soluble oral patch.

FIG. 1b shows a top view of the same oral patch.

FIG. 2 shows, in representational form, the structure of the solid, porous network, including the pores.

### DETAILED DESCRIPTION

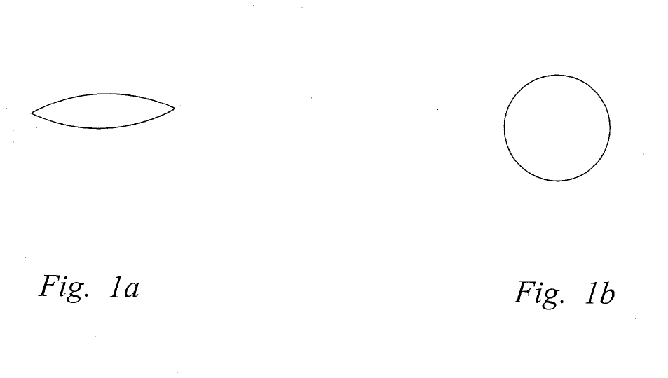

FIG. 1 shows a preferred shape for the oral patch. It has a feel and texture like gummy candies. It is made with slowly dissolving hydrocolloids so that that it typically lasts in the mouth for at least one to six hours. The patch can be formed in the shape of a tablet or a lozenge or a wafer or any other desired shape. A preferred shape is a thin lentil as shown in FIG. 1a.

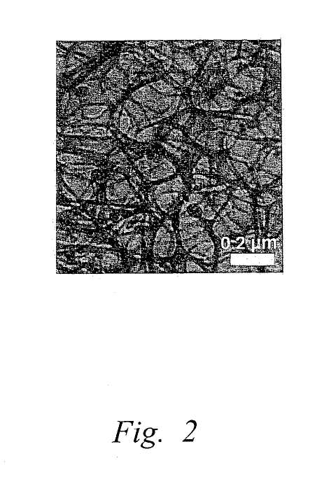

FIG. 2 shows, in representational form, the structure of the solid, porous network, including the pores. To practice this invention, a requirement for the network is that it remains a solid, rather than melting, at human mouth temperatures. So that the oral patch will slowly erode, the network should be made of a material with a low to moderate rate of disintegration in warm saliva. If the network does not erode fast enough, medication will be drawn out of the network faster than the network erodes and, although a portion of the oral patch remains in the mouth, it will no longer be doing any good. The hydrophilic polymer that, along with water and medication, fills pores of the network helps to slow the loss of medication from within pores of the network. Being a polymer, its molecules are long and they tend to be entangled by the network. The polymer molecules, in turn, along with the network structure, tend to entangle molecules of the medication. To obtain greater entanglement, molecules of the medication may be weakly chemically bound, such as by cross-linking, to molecules of the hydrophilic polymer or the network or both.

To understand by analogy how the porous network filled with a polymer that is liquid at mouth temperatures becomes very sticky without disintegrating, imagine a fish net bag filled with linguini Alfredo. When the linguini Alfredo is cold, such as when just removed from a refrigerator, the Alfredo sauce is congealed and the entire structure is not very sticky. Then put it in a microwave oven. The Alfredo sauce melts and becomes quite sticky. By itself, the fishnet bag is not sticky. But, the holes are large enough that strands of linguini covered with sauce will bulge out of the holes. When warm, the entire structure, if thrown against a wall, would probably stick, yet the bag keeps it all together as one piece. The strands of linguini are like the long molecules of a polymer that is liquid at mouth temperatures. Their length keeps them from easily falling out of the fish net bag.

Many different compositions can be used to form the network. For ease of manufacturing, it is convenient if the network is comprised of a thermo gel having a melting temperature higher than human mouth temperatures. This allows the entire mixture to be a liquid at temperatures far above human mouth temperatures and allows the network to be formed by cooling the mixture such that the thermo gel forms the desired network by a gelation process. The temperature at which the gel forms can be lower than human mouth temperatures, provided the temperature at which it melts again is higher than human mouth temperatures.

Readily available materials that form such a gel include agar, in various forms, carrageenan, in most of its forms, particularly kappa carrageenan, konjac gum, locust bean gum, and xanthan gum. All of these materials form a thermo gel that is sufficiently elastic or plastic or a combination thereof for the network to feel soft in the human mouth if it is adequately hydrated. If water is dried out of the network, it will become hard and will produce an unattractive feel when placed in contact with sensitive tissues, such as canker sores. To prevent the network from drying out, it may be packaged with a hermetic seal or a non-evaporating plasticizer, such as glycerol (glycerin) may be added. However, the more glycerol is added the less adherent the oral patch will be.

Synthetic hydrogels may be used for either the network that does not melt at mouth temperatures or the adherent, liquid polymer. Protein-based hydrogels are usually prepared using proteins extracted from natural sources, but they may be synthesized, such as with diblock copolypeptide amphiphiles, as taught by Nowak, et. al, “Rapidly Recovering Hydrogel Scaffolds From Self-Assembling Diblock Copolypeptide Amphiphiles”. Nowak, A. P.; Breedveld, V.; Pakstis, L.; Ozbas, B.; Pine, D. J.; Pochan, D.; Deming, T. J. Nature, 2002, 417, 424-428. The use of synthetic materials allows adjustment of copolymer chain length and composition. Synthetic hydrogels may also be made from polysaccharides and synthetic block copolymers which form thermoreversible gels and allow the solubilisation of hydrophobic medications for controlled release, as taught by Williams, P A, at the Centre for Water Soluble Polymers, North East Wales Institute, Plas Coch, Mold Road, Wrexham, Wales.

Instead of forming the network with a true hydrogel, the network may be formed with a complex carbohydrate, such as cellulose, pectin, starch, maltodextrin or other polysaccharides. Forming of hydrated network structures out of such materials is well known in the candy making industry for making gummy candies. Or the network may be formed with a combination of a true hydrogel and a complex carbohydrate.

The most crucial ingredient for the adherent oral patch is a hydrophilic polymer that is liquid at human mouth temperatures located within pores of the network. Gelatin rendered from animal protein, such as from pork skin or cattle hooves or from fish, serves very well as this ingredient. These gelatins are graded according to “bloom strength” which refers to the strength of the gel that is formed. Gelatin with a higher bloom strength is preferred for the adherent oral patch because it also has a higher viscosity in liquid form. The high viscosity in liquid form prevents the gelatin molecules from escaping the network substantially faster than the network erodes, and the high viscosity better retains the medication for slow release. The highest commercially available bloom strength, 250, is preferred.

The adherent oral patch is suitable for use with all of the medications mentioned in U.S. Pat. No. 6,139,861 issued to Friedman, including steroids, such as a glucocorticoid steroids, and non-steroidal anti-inflammatory drugs such as naproxen sodium, ibuprofen, acetaminophen, and ketoprofen. The medication may also be an antimicobial, such as an anti-fungal for treatment of candida organisms (thrush), such as nystatin, clotrimazole, miconazole, or fluconazole. The medication may be intended for treatment of canker sores (aphthous ulcers), including pharmaceutical antibiotics such as tetracycline, penicillin, or amoxicillin, or other canker sore treatment medications such as deglycyrrhizinated licorice root extract (DGL) or amlexanox.

If the network is formed of a hydrogel as described above, the oral patches may be manufactured by processes well known in the candy making industry. The process is to form a well-hydrated mixture at temperatures just below the boiling temperature of water so that water does not boil off and yet the hydrogels are fully activated for gelling when the product is cooled. In this process, the network can be formed of a combination of a true hydrogel such as xanthan gum with locust bean gum or with konjac gum and a complex carbohydrate such as cellulose or pectin or starch. For the medication deglycyrrhizinated licorice root extract (DGL), an effective ratio by weight is 56% water, 16% gelatin, 11% DGL, 10% cellulose, 4.8% glycerol, and 2.2% gums such as kappa carrageenan or xanthan gum plus locust bean gum or konjac gum heated to 200 degrees F.

The hot mixture is poured or squirted into molds. The molds be may open top molds or closed molds. Open top molds may be formed by pressing a plug into powdered starch such as cornstarch or may be formed in a tray for packaging the products such as thermo formed PVC or PET or a cold press laminate of aluminum and PVC with a thin layer of polyamide for strength. Closed molds may be used such as in an injection-molding machine. Because the mixture typically requires about 2 hours to form a strong enough oral patch for de-molding, it is preferable to intermittently move trays of two-part molds, upper and lower, under pump depositer injector nozzles. The nozzles fit into holes in the upper mold located at the center of each oral patch. After de-molding, the upper molds are used again for another batch. The lower molds may be plastic lined, in which case the plastic becomes a part of the final packaging. A suitable size for each oral patch is 0.8 grams poured into the mold.

If the oral patches are deposited in powdered starch, the starch absorbs some of the excess water and the oral patches are further dried in a drying room before being removed from the starch, packaged in a hermetic seal, and sterilized with gamma radiation or heat and pressure in a retort.

If the oral patches are deposited in molds formed in a tray, the tray is stored in a drying room until the oral patches lose a suitable amount of moisture. A suitable method of drying in trays is to expose them without convection to room temperature and humidity for 3 days or, with convection, for 24 hours. In the drying process, the oral patches lose about 47% of their weight, so an oral patch that started at 0.8 grams poured into the mold becomes 0.42 grams. The trays are then sealed with a film or foil lid that is adhered by conventional heat-sealing techniques and the entire package is sterilized with gamma radiation or heat and pressure in a retort.

For most applications, most users prefer that the oral patches be medium dry to dry. With this starting dryness, the oral patches are more adherent and have more integrity so they can be removed for talking or eating and then replaced. The only drawback to this dryness is that the oral patch becomes hard when it dries, giving the oral patch a less soothing feel. It is also less conforming and therefore does not stick well to hard surfaces such as guns and teeth. When the oral patch is placed in contact with delicate tissue, such as a large canker sore, most users prefer that the oral patch be moist and soft. Thus, it is preferable to package the oral patches with a film that allows moisture to pass so moisture can easily be added to or removed from the oral patches without removing them from the packaging. If the packaging film is a barrier to germs, this allows the oral patches to remain sterile and not grow mold even when they are moist. Effective films are cellophane, polystyrene, poybutadiene, polyamide, Tyvek (matted polyethylene threads) and expanded films such as Goretex. Polyamide with a thickness of 0.7 mil to 1.0 mil is effective. Allowing such a package to sit for a day or two with a few drops of water on the package is sufficient to hydrate the oral patch inside. Conversely, allowing the package to sit on a shelf in a dry room for one to three days is sufficient to dry out the oral patch.

While particular embodiments of the invention have been described above, the scope of the invention should not be limited by the above descriptions but rather limited only by the following claims.

1. An adherent oral patch for delivering medication in a human mouth over time, comprising:
(a) a porous network with a low to moderate rate of disintegration in saliva formed as a unitary solid structure that remains a solid at human mouth temperatures;
(b) molecules of a medication located within pores of the network; and
(c) molecules of a hydrophilic polymer that is liquid at human mouth temperatures, located within pores of the network.

2. The oral patch of claim 1 wherein the network is hydrophilic.

3. The oral patch of claim 1 wherein the network is comprised of a thermogel having a melting temperature higher than human mouth temperatures.

4. The oral patch of claim 3 wherein the thermogel is elasto-plastic.

5. The oral patch of claim 4 wherein the thermogel is a mixture of konjac gum and xanthan gum dissolved in hot water and then cooled to form an elasto-plastic gel.

6. The oral patch of claim 2 wherein the network is comprised of at least one hydrogel selected from the group of konjac gum, xanthan gum, locust bean gum, agar and carrageenan.

7. The oral patch of claim 1 wherein the network is comprised of complex carbohydrate.

8. The oral patch of claim 7 wherein the network is comprised of cellulose.

9. The oral patch of claim 7 wherein the network is comprised of at least one complex carbohydrate selected from the group of pectin, potato starch, rice starch, cornstarch, wheat starch, or maltodextrin.

10 The oral patch of claim 1 wherein the network is comprised of amino acids.

11. The oral patch of claim 10 wherein the amino acids are peptide-based hydrogels.

12. The oral patch of claim 1 wherein the hydrophilic polymer forms a thermoreversible gel that is a gel at room temperatures and a liquid at human mouth temperatures.

13. The oral patch of claim 12 wherein the hydrophilic polymer is gelatin rendered from animal tissue.

14. The oral patch of claim 1 wherein the hydrophilic polymer is comprised of protein.

15. The oral patch of claim 14 wherein the hydrophilic polymer is gelatin rendered from animal tissue.

16. The oral patch of claim 1 wherein the medication is a steroid.

17. The oral patch of claim 16 wherein the steroid is a glucocorticoid steroid.

18. The oral patch of claim 1 wherein the medication is a non-steroidal anti-inflammatory.

19. The oral patch of claim 1 wherein the medication is an antibiotic.

20. The oral patch of claim 19 wherein the medication is an anti-fungal.

21. The oral patch of claim 1 wherein the medication is deglycyrrhizinated licorice (DGL).

22. The oral patch of claim 1 wherein the medication is L-lysine amino acid.

23. The oral patch of claim 1 surrounded by packaging material comprising a germ barrier moisture-passing film.

24. A method for manufacturing an adherent oral patch for delivering topical medication in a human mouth over time, comprising:
(a) mixing a mixture comprising the medication, ingredients for forming a hydrophilic porous network with a low to moderate rate of disintegration in saliva that remains a solid at human mouth temperatures, molecules of a hydrophilic polymer that is liquid at human mouth temperatures, and water;
(b) heating the mixture; and
(c) cooling the mixture, thereby causing the ingredients for forming a network to form a hydrophilic porous network as a unitary solid structure having the medication and the hydrophilic polymer within its pores.

25. The method of claim 24 wherein the network is formed by a process of gelation resulting from the cooling step.

26. The method of claim 24 where, in the process of cooling, the hydrophilic polymer forms a thermoreversible gel that is a gel at room temperatures and a viscous liquid at human mouth temperatures.

27. The method of claim 24 wherein the hydrophilic polymer is comprised of protein.

28. The method of claim 27 wherein the hydrophilic polymer is gelatin rendered from animal tissue.

29. The method of claim 24 further comprising, between the heating step and the cooling step, depositing hot mixture into a mold and the cooling step is performed with the mixture in the mold.

30. The method of claim 29 wherein the mold is formed in powdered starch.

31. The method of claim 29 wherein the mold is rigid and comprises at least one layer selected from the group of plastic and aluminum.

32. The method of claim 25 further comprising packaging the oral patch in surrounding material that comprises a germ barrier moisture-passing film.

33. A moisture adjustable packaged oral patch, comprising:
(a) an oral patch surrounded by packaging material comprising a germ barrier moisture-passing film.

34. The packaged oral patch of claim 33 wherein the film is selected from a genus of germ barrier, moisture-passing films comprising cellophane, polystyrene, polybutadiene, polyamide, Tyvek (matted polyethylene threads) and expanded films including Goretex

35. The packaged oral patch of claim 33 wherein the film is polyamide.

36. A soft, adherent oral patch, comprising:
(a) a porous network with a low to moderate rate of disintegration in saliva comprising a high temperature hydrogel having a melting temperature higher than human mouth temperatures formed as a unitary solid structure;
(b) molecules of a medication located within pores of the network; and
(c) molecules of a low temperature hydrogel that melts at human mouth temperatures and gels at temperatures below mouth temperatures but above clothes pocket temperatures, located within pores of the network.

37. The oral patch of claim 36 wherein the medication is a steroid.

38. The oral patch of claim 36 wherein the medication is a non-steroidal anti-inflammatory.

39. The oral patch of claim 36 wherein the medication is an antibiotic.

40. The oral patch of claim 39 wherein the medication is an anti-fungal.

41. The oral patch of claim 36 wherein the medication is deglycyrrhizinated licorice (DGL).

42. The oral patch of claim 36 wherein the hydrophilic polymer is comprised of protein.

43. The oral patch of claim 42 wherein the low temperature hydrogel is gelatin rendered from animal tissue.

44. The oral patch of claim 36 surrounding by packaging material comprising a germ barrier moisture-passing film.
